(19) United States
(12) Patent Application Publication
(10) Pub. No.: US 2012/0172589 A1
(43) Pub. Date: Jul. 5, 2012

(54) PROCESS FOR THE SYNTHESIS OF IVABRADINE AND ADDITION SALTS THEREOF WITH A PHARMACEUTICALLY ACCEPTABLE ACID

(75) Inventors: Jean-Louis Peglion, Le Vesinet (FR); Pascal Caignard, Epinay-Sur-Sene (FR); Jean-Michel Lerestif, Yvetot (FR); Jean-Pierre Lecouve, Le Havre (FR)

(73) Assignee: LES LABORATOIRES SERVIER, Suresnes Cedex (FR)

(21) Appl. No.: 13/496,326

(22) PCT Filed: Sep. 17, 2010
(86) PCT No.: PCT/FR10/00625

§ 371 Date: Mar. 15, 2012

(30) Foreign Application Priority Data
Sep. 18, 2009 (FR) 09/04463

(51) Int. Cl. C07D 223/16; C07C 309/24; C07C 309/49; C07C 217/74

(52) U.S. Cl. 540/523; 564/428; 562/43; 562/66

(57) ABSTRACT

Process for the synthesis of ivabradine of formula (I):

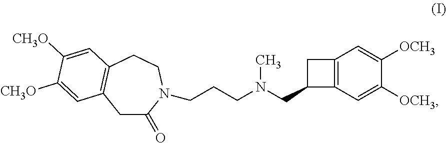

and addition salts thereof with a pharmaceutically acceptable acid.

The present invention relates to a process for the synthesis of ivabradine of formula (I):

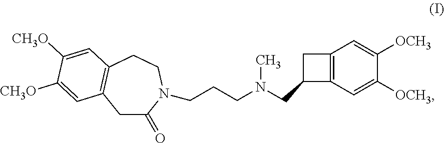

or 3-{3-[{[(7S)-3,4-dimethoxybicyclo[4.2.0]octa-1,3,5-trien-7-yl]methyl}(methyl)amino]-propyl}-7,8-dimethoxy-1,3,4,5-tetrahydro-2H-3-benzazepin-2-one,
addition salts thereof with a pharmaceutically acceptable acid, and hydrates thereof.

Ivabradine, and its addition salts with a pharmaceutically acceptable acid, and more especially its hydrochloride, have very valuable pharmacological and therapeutic properties, especially bradycardic properties, making those compounds useful in the treatment or prevention of various clinical situations of myocardial ischaemia such as angina pectoris, myocardial infarct and associated rhythm disturbances, and also in various pathologies involving rhythm disturbances, especially supraventricular rhythm disturbances, and in heart failure.

The preparation and therapeutic use of ivabradine and its addition salts with a pharmaceutically acceptable acid, and more especially its hydrochloride, have been described in the European patent specification EP 0 534 859.

That patent specification describes the synthesis of ivabradine hydrochloride starting from the compound of formula (II):

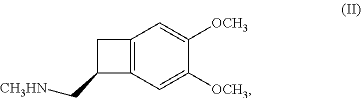

which is reacted with the compound of formula (III):

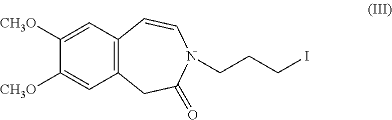

to yield the compound of formula (IV):

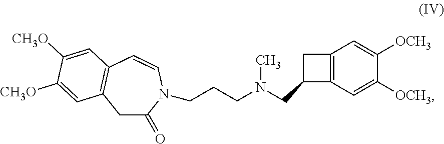

the catalytic hydrogenation of which yields ivabradine, which is then converted into its hydrochloride.

The disadvantage of that synthesis route is that it results in ivabradine in a yield of less than 17% over the three steps as a whole.

In view of the pharmaceutical value of this compound, it has been important to be able to obtain it by an effective synthesis process resulting in ivabradine in a good yield.

The international application WO 2008/065681 discloses a method of preparing ivabradine hydrochloride in which the compound of formula (II):

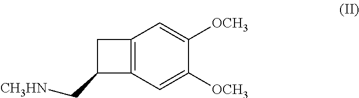

is reacted with 1-bromo-3-chloropropane in the presence of potassium carbonate to yield the compound of formula (V):

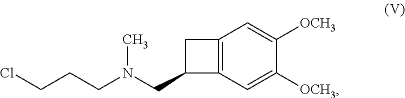

which, without being purified beforehand, is reacted with the compound of formula (VI):

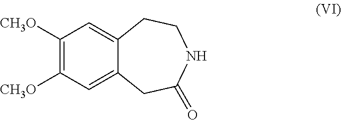

in the presence of potassium tert-butylate in dimethyl sulphoxide,
to yield ivabradine of formula (I), which, without being purified beforehand, is then converted into its hydrochloride.

The overall yield of that synthesis route is not mentioned in the application WO 2008/065681.

The Applicant has found, however, that it is not possible to prepare ivabradine hydrochloride by reproducing the procedure described in the application WO2008/065681.

The present invention relates to a process for the synthesis of ivabradine of formula (I), addition salts thereof with a pharmaceutically acceptable acid and hydrates thereof:

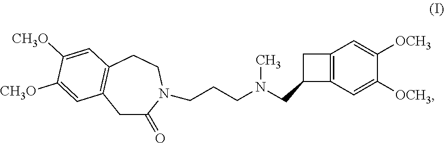

which process is characterised in that the compound of formula (VIII):

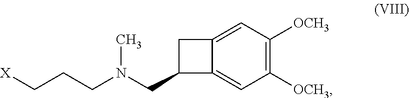

wherein X represents a halogen atom, a mesylate group or a tosylate group,
is subjected to an alkylation reaction with the compound of formula (IX):

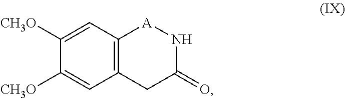

wherein A represents H₂C—CH₂ or HC═CH,
in the presence of a base,
in an organic solvent,
to yield the compound of formula (VII):

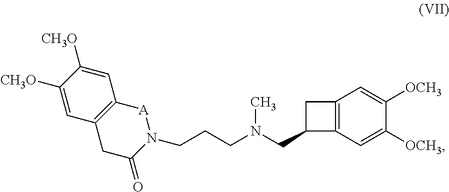

wherein A is as defined hereinbefore,
and then,
in the case where A represents H₂C—CH₂, ivabradine of formula (I), a particular case of the compounds of formula (VII) and product of the alkylation reaction of the compound of formula (VIII) with the compound of formula (IX), is isolated and purified and then may be converted into its addition salts with a pharmaceutically acceptable acid selected from hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, benzenesulphonic acid and camphoric acid, and into hydrates thereof,
in the case where A represents CH═CH, the compound of formula (IV), product of the alkylation reaction of the compound of formula (VIII) with the compound of formula (IX), is subjected to a catalytic hydrogenation reaction to yield ivabradine of formula (I), which is isolated and purified and then may be converted into its addition salts with a pharmaceutically acceptable acid selected from hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, benzenesulphonic acid and camphoric acid, and into hydrates thereof.

Among the bases that may be used to carry out the alkylation reaction of the compound of formula (VIII) with the compound of formula (IX) there may be mentioned, without implying any limitation, sodium hydride, potassium tert-butylate, sodium methanolate, potassium hydroxide, sodium hydroxide, potassium carbonate or caesium carbonate.

Preference is given to the base used to carry out the alkylation reaction of the compound of formula (VIII) with the compound of formula (IX) being potassium tert-butylate.

Among the solvents that may be used to carry out the alkylation reaction of the compound of formula (VIII) with the compound of formula (IX) there may be mentioned, without implying any limitation, tetrahydrofuran, 1,4-dioxane, dimethyl sulphoxide, tert-butanol, N,N-dimethylformamide, N,N-dimethyl acetamide or N-methylpyrrolidone.

Preference is given to the solvent used to carry out the alkylation reaction of the compound of formula (VIII) with the compound of formula (IX) being dimethyl sulphoxide.

The compounds of formula (VIIIa), particular cases of the compounds of formula (VIII) wherein X represents a bromine or iodine atom, a mesylate group or a tosylate group, are new products which are useful as synthesis intermediates in the chemical or pharmaceutical industry, especially in the synthesis of ivabradine, addition salts thereof with a pharmaceutically acceptable acid and hydrates thereof, and as such they form an integral part of the present invention.

The Examples hereinbelow illustrate the invention.

### LIST OF ABBREVIATIONS USED

### DMF: N,N-dimethylformamide

DMSO: dimethyl sulphoxide
IR: infrared

The melting points (m.p.) were measured using a Kofler block.

### EXAMPLE 1

### 3-{3-[{[(7S)-3,4-dimethoxybicyclo[4.2.0]octa-1,3,5-trien-7-yl]methyl}-(methyl)amino]propyl}-7,8-dimethoxy-1,3,4,5-tetrahydro-2H-3-benzazepin-2-one hydrochloride

### Step 1: 3-chloro-N-{[(7S)-3,4-dimethoxybicyclo[4.2.0]octa-1,3,5-trien-7-yl]methyl}-N-methylpropan-1-amine

Potassium carbonate (9.9 g; 72 mmol) is added to a solution of (1S)-4,5-dimethoxy-1-(methylaminomethyl)benzocyclobutane (10 g; 48 mmol) in 1-bromo-3-chloropropane (30 mL). After being in contact overnight at ambient temperature, the reaction mixture is poured into a mixture of distilled water (30 mL) and dichloromethane (30 mL). After separation, the organic phase is extracted with 2N HCl, and then the aqueous phase is brought to pH 9-10 after treatment with 28% aqueous ammonia solution. The organic phase obtained after extraction of the basic aqueous phase with dichloromethane is washed with distilled water and then dried over MgSO₄. The residue obtained after concentration under reduced pressure is purified by chromatography over silica (dichloromethane/ethyl acetate: 80/20) and 7.7 g of the title product are obtained in the form of crystals.

Yield=56%

m.p.=42-45° C.

### Step 2: 3-{3-[{[(75)-3,4-dimethoxybicyclo[4.2.0]octa-1,3,5-trien-7-yl]methyl}(methyl)-amino]propyl}-7,8-dimethoxy-1,3,4,5-tetrahydro-2H-3-benzazepin-2-one

Potassium tert-butylate (1.49 g; 13.3 mmol) is added to a solution of 7,8-dimethoxy-1,3,4,5-tetrahydro-2H-3-benzazepin-2-one (2.6 g; 11.75 mmol) in DMSO at ambient temperature. After being in contact for 1 hour at ambient temperature there is added a solution of 3.5 g (12.3 mmol) of the product obtained in the Step above in DMSO (4.7 mL). After being in contact overnight at ambient temperature, the reaction mixture is poured into distilled water (100 mL), and then the aqueous phase is extracted with ethyl acetate. The combined organic phases are washed with distilled water and then dried over MgSO₄. After concentration under reduced pressure, the residue obtained is purified by chromatography over silica (dichloro-methane/ethanol/NH₄OH 28%: 95/5/0.5) and 3.65 g of the title product are obtained in the form of an oil (HPLC purity: 98%) and used in the next Step.

Yield=66%

IR (pure): ν=2787, 1645, 1246-1206, 832 cm⁻¹.

### Step 3: 3-{3-[{[(75)-3,4-dimethoxybicyclo[4.2.0]octa-1,3,5-trien-7-yl]methyl}(methyl)-amino]propyl}-7,8-dimethoxy-1,3,4,5-tetrahydro-2H-3-benzazepin-2-one hydrochloride

1N ethereal hydrogen chloride (12 mL) is added to a solution of the product obtained in the Step above (3.6 g; 7.6 mmol) in acetonitrile (40 mL). After being in contact overnight, the suspension is cooled to 0° C. and then filtered. 3 g of the title product are obtained in the form of white crystals.

Yield=78%

m.p.=125-128° C.

### EXAMPLE 2

### 3-{3-[{[(7S)-3,4-dimethoxybicyclo[4.2.0]octa-1,3,5-trien-7-yl]methyl}-(methyl)amino]propyl}-7,8-dimethoxy-1,3,4,5-tetrahydro-2H-3-benzazepin-2-one hydrochloride

### Step 1: 3-chloro-N-{[(7S)-3,4-dimethoxybicyclo[4.2.0]octa-1,3,5-trien-7-yl]methyl}-N-methylpropan-1-amine

The title product is prepared by following the procedure described in Step 1 of Example 1.

### Step 2: 3-{3-[{[(7S)-3,4-dimethoxybicyclo[4.2.0]octa-1,3,5-trien-7-yl]methyl}(methyl)-amino]propyl}-7,8-dimethoxy-1,3-dihydro-2H-3-benzazepin-2-one

Potassium tert-butylate (1.7 g; 15.15 mmol) is added to a solution of 7,8-dimethoxy-1,3-dihydro-2H-3-benzazepin-2-one (2.94 g; 13.4 mmol) in DMSO (12 mL) at ambient temperature. After being in contact for 30 minutes at ambient temperature there is added a solution of 4 g (14.1 mmol) of the product obtained in the preceding Step in DMSO (10 mL). After being in contact overnight at ambient temperature, the reaction mixture is poured into distilled water (100 mL) and then the aqueous phase is extracted with ethyl acetate. The combined organic phases are washed with distilled water and then dried over MgSO₄. After concentration under reduced pressure, 6.2 g of the title product are obtained in the form of an oil (HPLC purity: 88%) and used in the next Step.

Yield=87%

IR (pure): ν=2788, 1656, 1510-1401, 836-760 cm⁻¹.

### Step 3: 3-{3-[{[(7S)-3,4-dimethoxybicyclo[4.2.0]octa-1,3,5-trien-7-yl]methyl}(methyl)-amino]propyl}-7,8-dimethoxy-1,3,4,5-tetrahydro-2H-3-benzazepin-2-one

In a 250-ml autoclave, 4 g of the product obtained in the Step above and 2 g of Pd(OH)₂ 20%, 50% wet, are added to a solution of ethanol (90 mL) and acetic acid (10 mL). After being in contact for 5 hours at ambient temperature under a hydrogen pressure of 5 bar, the reaction mixture is filtered over Celite. The residue obtained after concentration under reduced pressure is taken up in dichloromethane (100 mL) and then washed with saturated aqueous sodium bicarbonate solution. The oil obtained after drying of the organic phase over MgSO₄ and then concentrating under pressure is purified by chromatography over silica (dichloromethane/ethanol/NH₄OH 28%: 95/5/0.5) and 2.6 g of the title product are obtained in the form of an oil.

Yield=74%

IR (pure): ν=2788, 1646, 1519-1461, 1245-1105 cm¹.

### Step 4: 3-{3-[{[(75)-3,4-dimethoxybicyclo[4.2.0]octa-1,3,5-trien-7-yl]methyl}(methyl)-amino]propyl}-7,8-dimethoxy-1,3,4,5-tetrahydro-2H-3-benzazepin-2-one hydrochloride

2.9N ethanolic hydrogen chloride (3 mL) is added to a solution of the product obtained in the Step above (2.6 g; 5.5 mmol) in acetonitrile (25 mL). After being in contact overnight, the suspension is filtered and 2.2 g of the title product are obtained in the form of white crystals.

Yield=79%

m.p.=123-125° C.

### COMPARISON EXAMPLE

### Reproduction of the Procedure Described in the Application WO 2008/065681

### Step 1: 3-chloro-N-{[(7S)-3,4-dimethoxybicyclo[4.2.0]octa-1,3,5-trien-7-yl]methyl}-N-methylpropan-1-amine

Potassium carbonate (9.9 g; 72 mmol) is added to a solution of (1S)-4,5-dimethoxy-1-(methylaminomethyl)-benzocyclobutane (10 g; 48 mmol) in 1-bromo-3-chloropropane (30 mL). After being in contact overnight at ambient temperature, the reaction mixture is poured into a mixture of distilled water (30 mL) and dichloromethane (30 mL). After separation, the organic phase is extracted with 2N HCl and then the aqueous phase is brought to pH 9-10 after treatment with 28% aqueous ammonia solution. The organic phase obtained after extraction of the basic aqueous phase with dichloromethane is washed with distilled water and then dried over MgSO₄. After concentration under reduced pressure, the title product is obtained in a crude yield of 82% by weight and with a purity of 56%. The crude reaction product still contains 40% (1S)-4,5-dimethoxy-1-(methylaminomethyl)-benzo-cyclobutane.

### Step 2: 3-{3-[{[(7S)-3,4-dimethoxybicyclo[4.2.0]octa-1,3,5-trien-7-yl]methyl}(methyl)-amino]propyl}-7,8-dimethoxy-1,3,4,5-dihydro-2H-3-benzazepin-2-one

Potassium tert-butylate (1.49 g; 13.3 mmol) is added to a solution of 7,8-dimethoxy-1,3,4,5-tetrahydro-2H-3-benzazepin-2-one (2.6 g; 11.75 mmol) in DMSO at ambient temperature. After being in contact for 1 hour at ambient temperature there is added a solution of 3.5 g (12.3 mmol) of the product obtained in the Step above in DMSO (4.7 mL). After being in contact overnight at ambient temperature, the reaction mixture is poured into distilled water (100 mL), and then the aqueous phase is extracted with ethyl acetate. The combined organic phases are washed with distilled water and then dried over MgSO₄. After concentration under reduced pressure, the title compound is obtained in a crude yield of 96.8% and with a purity of 55%.

### Step 3: 3-{3-[{[(7S)-3,4-dimethoxybicyclo[4.2.0]octa-1,3,5-trien-7-yl]methyl}(methyl)-amino]propyl}-7,8-dimethoxy-1,3,4,5-tetrahydro-2H-3-benzazepin-2-one hydrochloride

To a solution of 5 g of the crude product obtained in the Step above in acetonitrile (15 mL) there is added a 6N solution of hydrochloric acid in isopropanol. After being in contact overnight at ambient temperature, the hydrochloride of the title compound did not precipitate out and therefore could not be isolated. Starting from the crude compound obtained in the previous Step, it was impossible to obtain the title product by following the procedure described in the application WO 2008/065681.

1-5. (canceled)

6. A process for the synthesis of ivabradine of formula (I), addition salts thereof with a pharmaceutically acceptable acid and hydrates thereof: 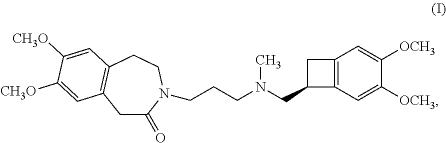
wherein a compound of formula (VIII): 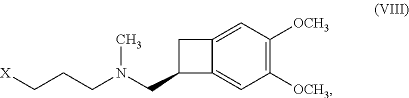
wherein X represents a halogen atom, a mesylate group or a tosylate group,
is subjected to an alkylation reaction with a compound of formula (IX): 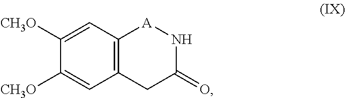
wherein A represents H₂C—CH₂ or HC═CH,
in the presence of a base,
in an organic solvent,
to yield the compound of formula (VII): 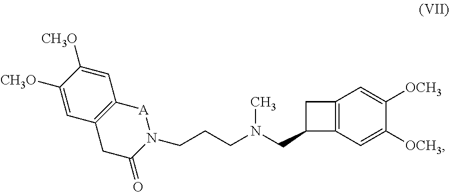
wherein A is as defined hereinbefore,
and then,
in the case where A represents H₂C—CH₂, ivabradine of formula (I) is isolated and purified and then may be converted into an addition salt with a pharmaceutically acceptable acid selected from hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, benzenesulphonic acid and camphoric acid, and into a hydrate thereof,
or
in the case where A represents CH═CH, the compound of formula (VII) is subjected to a catalytic hydrogenation reaction to yield ivabradine of formula (I), which is isolated and purified and then may be converted into an addition salt with a pharmaceutically acceptable acid selected from hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, benzenesulphonic acid and camphoric acid, and into a hydrate thereof.

7. The process according to claim 6, wherein the base used to carry out the alkylation reaction of the compound of formula (VIII) with the compound of formula (IX) is selected from sodium hydride, potassium tert-butylate, sodium methanolate and potassium hydroxide, sodium hydroxide, potassium carbonate and caesium carbonate.

8. The process according to claim 6, wherein the base used to carry out the alkylation reaction of the compound of formula (VIII) with the compound of formula (IX) is potassium tert-butylate.

9. The process according to claim 6, wherein the solvent used to carry out the alkylation reaction of the compound of formula (VIII) with the compound of formula (IX) is selected from tetrahydrofuran, 1,4-dioxane, dimethyl sulphoxide, tert-butanol, N,N-dimethylformamide, N,N-dimethyl acetamide and N-methylpyrrolidone.

10. A compound selected from those of formula (VIIIa): 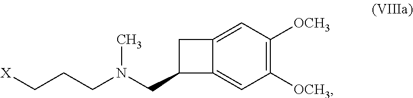
wherein X represents a bromine or iodine atom, a mesylate group or a tosylate group.
